(19) United States
(12) Patent Application Publication
(10) Pub. No.: US 2010/0087525 A1
(43) Pub. Date: Apr. 8, 2010

(54) STEREOSELECTIVE ENZYMATIC SYNTHESIS OF (S) OR (R)-ISO-BUTYL-GLUTARIC ESTER

(76) Inventors: Lilach Hedvati, Ein Shemer (IL); Greta Sterimbaum, Rishon Letzhion (IL); Yuriy Raizi, Natanya (IL); Rahamin Aminov, Natanya (IL)

Correspondence Address: KENYON & KENYON LLP, ONE BROADWAY, NEW YORK, NY, 10004, US

(21) Appl. No.: 12/490,025

(22) Filed: Jun. 23, 2009

Related U.S. Application Data
(60) Provisional application No. 61/074,903, filed on Jun. 23, 2008.
(60) Provisional application No. 61/137,738, filed on Jul. 31, 2008.

(51) Int. Cl. A61K 31/225; C12P 7/62; A61P 25/08

(52) U.S. Cl. 514/547; 435/135

(57) ABSTRACT

The present invention relates to a stereoselective enzymatic synthesis of (S) or (R)-iso-butyl-glutaric ester, an intermediate of S-Pregabalin.

### CROSS-REFERENCE TO RELATED APPLICATIONS

This application claims the benefit of U.S. Provisional Application Nos. 61/074,903, filed Jun. 23, 2008; and 61/137,738, filed Jul. 31, 2008, hereby incorporated by reference.

### FIELD OF THE INVENTION

The present invention relates to a stereoselective enzymatic synthesis of (S) or (R)-iso-butyl-glutaric ester, an intermediate of S-Pregabalin.

### BACKGROUND OF THE INVENTION

(S)-Pregabalin, (S)-(+)-3-(aminomethyl)-5-methylhexanoic acid, a compound having the following chemical structure,

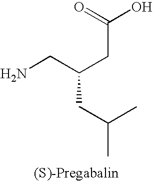

is a γ-amino butyric acid or (S)-3-isobutyl (GABA) analogue. (S)-Pregabalin has been found to activate GAD (L-glutamic acid decarboxylase). (S)-Pregabalin has a dose dependent protective effect on-seizure, and is a CNS-active compound. (S)-Pregabalin is useful in anticonvulsant therapy, due to its activation of GAD, promoting the production of GABA, one of the brain's major inhibitory neurotransmitters, which is released at 30 percent of the brains synapses. (S)-Pregabalin has analgesic, anticonvulsant, and anxiolytic activities.

Preparation of (S)-Pregabalin as disclosed in International Publication No. WO 2007/139933 (“WO '933”) is done by preparing (R)-(+)-3-(carbamoylmethyl)-5-methylhexanoic acid (“R—CMH”) or a salt thereof by asymmetric synthesis of (S)-iso-butyl-glutaric ester as illustrated in the following scheme:

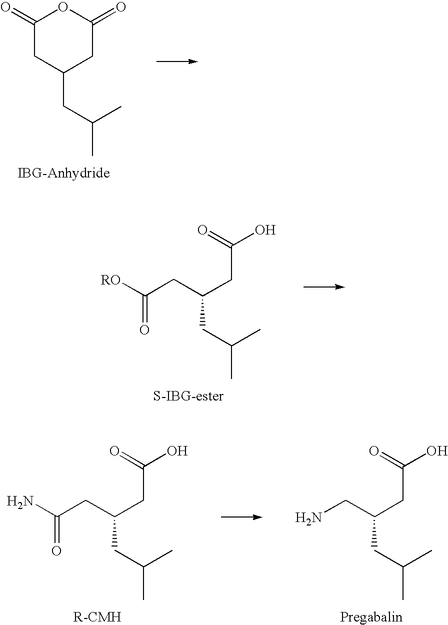

and then converting R—CMH to S-Pregabalin

In WO 2007/143113 (“WO '113”), intermediates of (S)-Pregabalin, such as R—CMH and (3S)-cyano-5-methylhexanoic acid (“(S)-pregabalin nitrile” or “S—PRG-nitrile”) are also prepared by kinetic resolution as described in the following scheme:

### 1. Hydrolysis

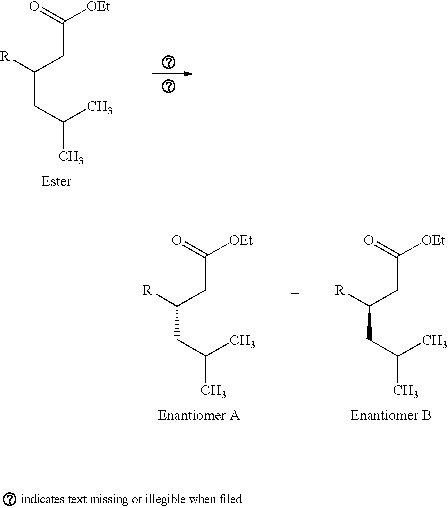

### 2. Esterification

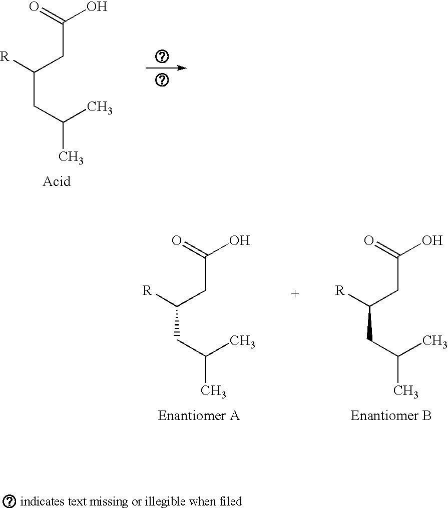

In the above processes the chirality is achieved either by an asymmetric reaction using a chiral agent (as reported in WO '933) or by kinectic resolution with an enzyme (as reported in WO '113) that reacts with only one enantiomer of the starting material.

The present invention offers routes for the preparation of (S)-iso-butyl-glutaric ester (“S—IBG-ester”) or (R)-iso-butyl-glutaric ester (“R—IBG-ester”) which is one of the first intermediates of S-Pregabalin (“S—PRG”), where a stereoselective enzymatic synthesis is utilized.

### SUMMARY OF THE INVENTION

In one embodiment, the invention encompasses a process for preparing (S)-iso-butyl-glutaric ester or (R)-iso-butyl-glutaric ester having the following formula,

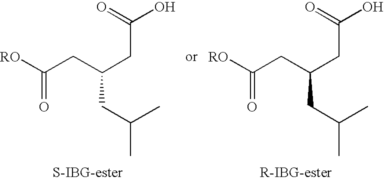

comprising: combining a suitable enzyme with a) 3-isobutylglutaric acid (“IBG acid”) of the following formula

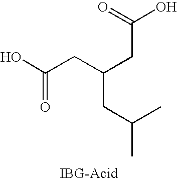

and an alcohol or an alkoxy donor that includes an OR group; or with b) 3-iso-butyl-glutaric diester (“IBG-diester”) of the following formula

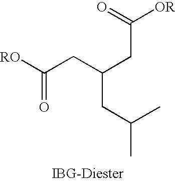

wherein the suitable enzyme is capable of stereoselectively esterifying IBG acid and stereoselectively hydrolyzing IBG-diester, respectively; and R is a C₁₋₇ hydrocarbyl group.

In another embodiment, the invention encompasses a process for preparing S-Pregabalin of the following formula

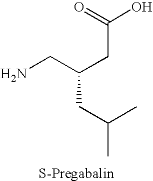

comprising: preparing S—IBG-ester or R—IBG-ester by the processes of the present invention and converting either one of them to S-Pregabalin.

In yet another embodiment, the invention encompasses a composition which comprises S—IBG-ester and between 0.1% and 5% area by HPLC of R—IBG-ester, based on the combined area % of said R—IBG-ester and S—IBG-ester as measured by HPLC.

In one embodiment, the invention encompasses a composition which comprises R—IBG-ester and between 0.1% and 5% area by HPLC of S—IBG-ester, based on the combined area % of said R—IBG-ester and S—IBG-ester as measured by HPLC.

In yet another embodiment, the invention encompasses the use of any one of the above compositions to prepare S-Pregabalin.

### DETAILED DESCRIPTION OF THE INVENTION

The present invention offers two routes for the preparation of (S)-iso-butyl-glutaric ester or (R)-iso-butyl-glutaric ester, which is one of the first intermediate of S-Pregabalin, where a stereoselective enzymatic synthesis is utilized.

In the stereoselective process of the present invention there is no need for optical resolution, and also there is no waste of the starting material as all of it reacts with the enzyme. Also, it is easy to isolate the product in high yield and enantiomeric excess from the reaction mixture. Further, the regeneration of the enzyme from the reaction mixture is simple, thus it can be used several times. Accordingly, the processes of the present invention are economical, environmental friendly, and suitable for industrial scale applications.

The two routes can be illustrated by the following scheme:
1. via Stereoselective hydrolysis:

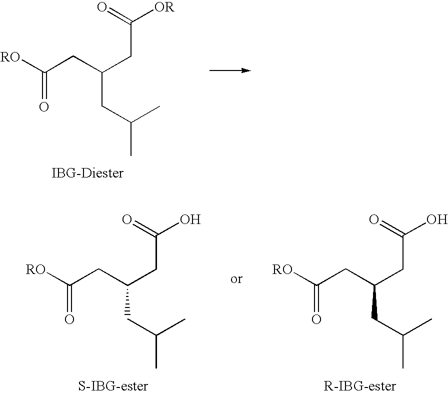

2. via Stereoselective esterification:

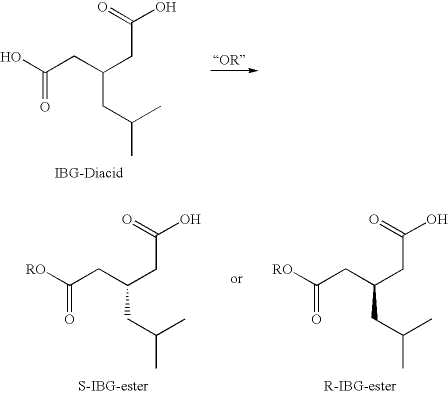

wherein a suitable enzyme, which is capable of stereoselectively esterifying MG acid and also stereoselectively hydrolyzing IBG-diester, depending on the conditions of the reaction, is used; and R is a C₁₋₇ hydrocarbyl group.

The obtained S—IBG-ester or R—IBG-ester can then be converted to S-Pregabalin via (R)-(+)-3-(carbamoylmethyl)-5-methylhexanoic acid (“R—CMH”) without the need to perform an optical resolution step in any stage.

In one embodiment, the invention encompasses one route for preparing S-MG-ester or R—IBG-ester, which may be illustrated by the following scheme:

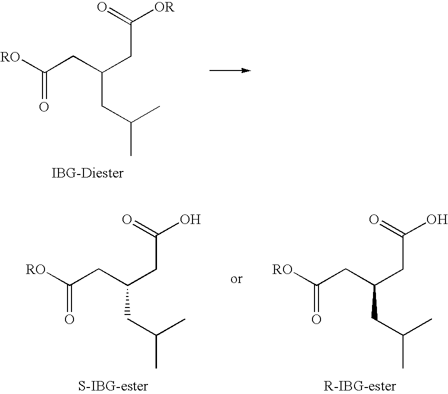

This route comprises combining a suitable enzyme with 3-iso-butyl-glutaric diester to obtain a reaction mixture, wherein the suitable enzyme is capable of stereoselectively hydrolyzing IBG diester; and R is a C₁₋₇ hydrocarbyl group.

Examples for a C₁₋₇ hydrocarbyl group includes, but is not limited to, benzyl, methyl, ethyl, propyl, vinyl or n-butyl. Preferably, the C₁₋₇ hydrocarbyl group is methyl, ethyl, propyl, vinyl or n-butyl. More preferably, the C₁₋₇ hydrocarbyl group is methyl, ethyl or n-butyl, and most preferably, the C₁₋₇ hydrocarbyl group is methyl.

Typically, the hydrolysis reaction is done in the presence of a buffer, thus providing the hydrolyzed product in the said reaction mixture, which is either S—IBG-ester or R—IBG-ester.

The starting IBG-ester can be prepared for example, according to the process disclosed in Example 1.

As mentioned above, the suitable enzyme is an enzyme capable of performing a stereoselective hydrolysis reaction of IBG-diester, thus providing the chiral S—IBG ester or R—IBG ester. Examples for such an enzyme include hydrolase, preferably, an esterase, lipase or protease.

Preferably, the esterase is selected from the group consisting of Esterase BS2 from bacillus species and Esterase BS3 from bacillus species.

Preferably, the lipase is selected from the group consisting of Lipase L-5, lipase from Aspergillus Oryzae, Lipase from Thermomyces lanuginosus, Lipase from Thermomyces lanuginosus mutant, Lipase mutant broad range from Thermomyces lanuginosus mutant, Lipase PS amono from Pseudomonas stutzeri, Lipase RS from Rhizopus spp., Lipase PF from Pseudomonas fluorescens, Lipase PC from Penicillium camenbertii, Lipase P1 from Pseudomonas cepacia, Lipase P2 from Pseudomonas cepacia, Lipase AN from Aspergillus niger, Lipase A from Candida Antartica, Lipase CA(A) from candida, Lipase CAL A from candida, Lipase AS1 from Alcaligenes spp., Lipase AS2 Alcaligenes spp, Lipase C2 from Candida cylindracea, Lipase C1 from Candida cylindracea, Lipase B from Candida Antartica, Lipase CA(B) from candida antartica, Lipase CAL B from candida, Lipase CAL BIM, Lipase from rhizomucor miehei, Lipase acceptin bulky substrate from fungal mutant Lipase broad range from fungal, Lipase broad range from fungal mutant, Lipase mucor sol from mucore miehei, Lipase mucor CF from mucore miehei, and Lipase MM from mucore miehei.

Preferably, the protease is selected from the group consisting of Protease alkaline from bacillus clausii, Protease alkaline and temperature stable from bacillus hludurans, Protease alkaline from bacillus licheniformis, Protease from bacillus licheniformis, Protease from fusarium oxysporum, and Protease from rhizomucor miehei.

More preferably, the hydrolase is Lipase acceptin bulky substrate from fungal mutant, Lipase from rhizomucor miehei, Lipase B from Candida Antartica, Lipase CA(B) from candida antartica, Lipase CA(A) from candida antartica, lipase from Aspergillus Oryzae, Esterase BS3 from bacillus species, Lipase mucor sol from mucore miehei, Lipase C2 from Candida cylindracea, Lipase P2 from Pseudomonas cepacia, or Esterase BS2 from bacillus species.

Preferably, Lipase B from Candida Antartica is a suitable enzyme for producing S—IBG-ester, more preferably, for producing S-methyl-IBG-ester.

Preferably, Lipase acceptin bulky substrate from fungal mutant, Lipase from rhizomucor miehei, lipase from Aspergillus Oryzae, Lipase CA(A) from candida antartica, Lipase C2 from Candida cylindracea, or Esterase BS3 from bacillus species are suitable enzymes for producing R—IBG-ester, more preferably, for producing R-methyl-IBG-ester.

Typically, the enzymes are used in a combination with a buffer. The buffer adjusts the pH of the reaction mixture to a pH level suitable for the enzymatic activity. Preferably, the buffer is present in an amount sufficient to provide a pH of about 6 to about 9. More preferably, the buffer is present in an amount sufficient to provide a pH of about 6.5 to about 8, and most preferably, the buffer is present in an amount sufficient to provide a pH of about 7.0 to about 7.8. Preferably, the buffer is K₂HPO₄ buffer or tris(hydroxymethyl)aminomethane (“TRIS”) buffer.

In some embodiments, 3-iso-butyl-glutaric diester is first combined with a buffer to obtain a mixture, to which the enzyme is added. In other embodiments, the buffer is first combined with the enzyme to obtain a mixture, to which IBG-diester is added, for example dropped-wise.

Optionally, a polar solvent is admixed with the mixture, this can increase the solubility of 3-iso-butyl-glutaric diester in the mixture. Preferably, the polar solvent is C₁₋₅ alcohol, more preferably, the C₁₋₅ alcohol is tert-pentanol.

Optionally, the mixture of 3-iso-butyl-glutaric diester in a buffer or the mixture of buffer with enzyme is cooled prior to the addition of the enzyme or the IBG-diester, respectively. Preferably, the cooling is performed to a temperature of about −3° C. to about 10° C. More preferably, the cooling is performed to a temperature of about −2° C.

For example, when preparing S—IBG-ester, the reaction can be done at a temperature of about (−10)° C. to about 40° C. Preferably, it can be done at a temperature of at about −3° C. to about 10° C. More preferably, it can be done at a temperature of about −2° C. to about 0° C. When preparing R—IBG-ester, the reaction can be done for example, at a temperature of about 20° C. to about 40° C., preferably, it can be done at a temperature of about 20° C. to about 30° C.

Further, the reaction is stirred at the above temperature, preferably, for about 1 hour to about 4 days. More preferably, it is stirred for about 40 to about 96 hours, during which the formation of S—IBG-ester or R—IBG-ester occurs.

Typically, the pH level (6-9) is maintained by an addition of a base, preferably, selected from the group consisting of alkaline hydroxide, carbonates, bicarbonates, and amines. More preferably, the base is sodium hydroxide, sodium carbonate or ammonia.

The hydrolysis process for preparing S—IBG-ester or R—IBG-ester can further comprises recovering the said S—IBG-ester or R—IBG-ester from the reaction mixture.

The recovery can be done, for example, by filtering the mixture to remove the enzyme, washing, acidifying the filtrate, extracting with an organic solvent and evaporating the combined extracts to obtain the product. Preferably, the filtrate is acidified to a pH of about 1.5 by an addition of an acid.

Typically, when the filtration is performed using an ultra filtration set up, it is preformed under a pressure. Optionally, the obtained filtrate is diluted and filtered again prior to acidifying it.

Optionally, the extract is dried under a drying agent, such as magnesium sulfate prior to evaporating it.

In another embodiment, the invention encompasses a second route for preparing S—IBG-ester or R—IBG-ester, which may be illustrated by the following scheme:

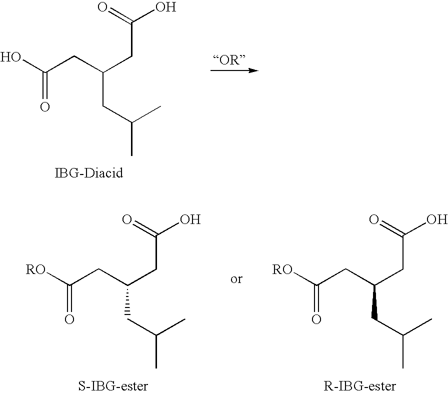

This route comprises combining a suitable enzyme with 3-isobutylglutaric acid and an alcohol or an alkoxy donor that includes an OR group, to obtain a reaction mixture, wherein the suitable enzyme is capable of stereoselectively esterifying IBG acid; and R is a C₁₋₇ hydrocarbyl group.

Examples for a C₁₋₇ hydrocarbyl group includes, but is not limited to, benzyl, methyl, ethyl, propyl, vinyl or n-butyl. Typically, the C₁₋₇ hydrocarbyl group is a C₁₋₅ hydrocarbyl group. Preferably, it is a C₁₋₄hydrocarbyl group, more preferably, it is methyl, ethyl, propyl, vinyl or n-butyl. Even more preferably, the hydrocarbyl group is methyl, ethyl or n-butyl, and most preferably, the hydrocarbyl group is methyl.

As used herein, the term “alkoxy donor” refers to a molecule that contains a labile OR moiety, i.e., such that the OR group can be transferred to another molecule, wherein R can be a hydrocarbyl group, as mentioned above. Examples for such molecules include, but are not limited to, esters, such as vinyl acetate, methyl acetate or ethyl acetate.

The starting IBG-Acid can be prepared for example, according to the process disclosed in U.S. Pat. No. 5,616,793.

Typically, the alcohol or alcohol donor is a C₁₋₇ alcohol or C₁₋₇ alkoxy donor. Preferably, the C₁₋₇ alcohol or alkoxy donor is benzyl alcohol, methanol, ethanol, propanol, vinyl acetate, methyl acetate or n-butanol, more preferably, the C₁₋₇ alcohol or alkoxy donor is methanol, ethanol, propanol, vinyl acetate, methyl acetate or n-butanol. Even more preferably, the C₁₋₇ alcohol or alkoxy donor is methanol, ethanol or n-butanol, and most preferably, the alcohol is methanol.

Preferably, the suitable enzyme is the same as those described above.

Typically, the reaction mixture containing the enzyme, IBG-Acid and the alcohol or alcohol donor further contains a solvent. Preferably, the amount of the enzyme is catalytic.

Suitable solvents include, but are not limited to, ketone, nitrile, aromatic hydrocarbon, ether and mixtures thereof. Preferably, the ketone is a C₃₋₆ ketone, more preferably, the C₃₋₆ ketone is acetone, methylethylketone (“MEK”), or methyl-isobutylketone (“MIBK”). Preferably, the nitrile is a C₂₋₄ nitrile, more preferably, the C₂₋₄ nitrile is acetonitrile (“ACN”). Preferably, the aromatic hydrocarbon is a C₆₋₉ aromatic hydrocarbon, more preferably, the C₆₋₉ aromatic hydrocarbon is toluene. Preferably, the ether is a C₃₋₇ ether, more preferably, the C₃₋₇ ether is diisopropylether

(“DIPE”), methyl-tertbutylether (“MTBE”) or tetrahydrofuran (“THF”). Most preferably, the solvent is either DIPE or toluene.

Preferably, the reaction is done at a temperature of about 5° C. to about 50° C., more preferably, it is done at a temperature of about 25° C. to about 37° C.

Preferably, the reaction is done for about 2 to about 96 hours, more preferably, it is done for about 2 to about 24 hours, during which the formation of S-MG-ester or R—IBG-ester occurs.

The obtained S—IBG-ester or R—IBG-ester is then recovered from the reaction mixture, for example as mentioned before.

Typically, the obtained or recovered S—IBG-ester is a composition which comprises for example, S—IBG-ester and between 0.1% to less than 5%, preferably, between 0.1% to 3% more preferably, between 0.1% to 1% area by HPLC of R—IBG-ester based on the combined area % of said R—IBG-ester and S—IBG-ester as measured by HPLC.

Further, the above composition contains for example, R—IBG-ester and between 95% and 99.9% area by HPLC, preferably, between 97% and 99.9%, more preferably, between 99% and 99.9% area by HPLC of S—IBG-ester, based on the combined area % of said R—IBG-ester and S—IBG-ester as measured by HPLC.

Typically, the obtained or recovered R-MG-ester is a composition which comprises for example, R—IBG-ester and between 0.1% and 5%, preferably, between 0.1% and 3% more preferably, between 0.1% and 1% area by HPLC of S—IBG-ester based on the combined area % of said R—IBG-ester and S—IBG-ester as measured by HPLC.

Further, the above composition contains for example, S—IBG-ester and between 95% and 99.9%, preferably, between 97% and 99.9%, more preferably, between 99% and 99.9% area by HPLC of R—IBG-ester, based on the combined area % of said R—IBG-ester and S—IBG-ester as measured by HPLC.

The S—IBG-ester or R-MG-ester obtained from the processes described herein can then be converted to S-Pregabalin. The conversion can be done by first transforming S—IBG ester or R—IBG-ester to R—CMH, for example by the process disclosed in International Publication No. WO 2007/139933, and then transforming R—CMH to S-Pregbalin for example by the process disclosed in US publication No. 2007/0073085.

The conversion can also be done by first transforming R—IBG ester to (R)-methyl 3-(carbamoylmethyl)-5-methylhexanoate, and then transforming it to S-Pregbalin, for example by a similar process as disclosed in International Publication No. WO 2008/118427.

Having described the invention with reference to certain preferred embodiments, other embodiments will become apparent to one skilled in the art from consideration of the specification. The invention is further defined by reference to the following examples describing in detail the preparation of (S)-iso-butyl-glutaric ester or (R)-iso-butyl-glutaric ester, an intermediate of S-Pregabalin. It will be apparent to those skilled in the art that many modifications, both to materials and methods, may be practiced without departing from the scope of the invention.

### Examples

HPLC Method for S-Methyl Ester and for R-Methyl Ester Optical Purity Determination

HPLC

| | |
|---|---|
| Column & | 2 DAICEL Chiralpak AD-H 250*4.6 mm P.N. 19325 |
| packing | (stationary phase is Amylose tris (3,5-dimethylphenyl- |
| | carbamate) coated on 5 micron silica-gel) |
| Eluent: | n-Hexane:n-Butanol:TFA |
| | 950:50:1 |
| Stop time: | 45 min |
| Flow: | 0.5 ml/min |
| Detector: | 212 nm. |
| Injection | 50 μl |
| volume: | |
| Diluent | 95:5 n-Hexane:n-Butanol |
| Column | 10° C. |
| temperature | |
| Auto sampler | 15° C. |
| temperature | |

### Sample Solution Preparation

About 40 mg of R-methyl ester sample was placed in a 10 ml volumetric flask, and was dissolved and diluted up to the volume with the diluent.

$$Calculations$$

$$\% S \text{-} IBG \text{-} Methyl Ester = \sum \frac{Area S \text{-} IBG \text{-} Methyl Ester}{\begin{matrix}Area S \text{-} IBG \text{-} Methyl Ester and \\ Area R \text{-} IBG \text{-} Methyl Ester\end{matrix}} \times 100$$

The optical purity results provided in the below examples are for one of the two enantiomers. Subtraction of the optical purity results of one enantiomer from 100% provides the amount of the second enantiomer in %.

### Example 1

### Preparation of iso-butyl-glutaric diester

To a 250 ml flask equipped with a magnetic stirrer and condenser were added iso-butyl glutaric methyl ester racemate (20 g, 99 mmol), methanol (80 ml) and H₂SO₄ 96% (1 ml). The mixture was stirred at reflux for 12 hours. The methanol was evaporated and the residue was diluted with toluene (100 ml). The organic phase was washed with NaOH 3% solution (3×35 ml). Then organic phase was evaporated to dryness to obtain iso-butyl-glutaric diester, as an oily material.

### Esterification:

### Example 2

### Esterification of IBG-acid by enzyme

To a 15 ml vial equipped with a magnetic stirrer were added IBG-acid (240 g), enzyme¹ (20 mg), solvent² (15 vol, 3.6 ml) and methanol (3 eq, 15 μl). The mixture was stirred at room temperature for 24 hours.
Enzymes¹: the following Lipase enzymes were used: CAL A L-5, CAL B, CAL B IM
Solvent²: diisopropyl ether, toluene.

### Example 3

### Hydrolysis of iso-butyl glutaric di-methyl ester (“IBG-dimethyl ester”)

To a 15 ml vial equipped with a magnetic stirrer were added iso-butyl-glutaric diester (240 mg), buffer K₂HPO₄ 0.1 M pH 7 and enzyme¹. The mixture was stirred at room temperature or at a higher temperature. The conditions and type of enzymes used are summarized at the table below:

| Type | source |
|---|---|
| Lipase A from C. antartica | candida antartica |
| Lipase B from C. antartica | candida antartica |
| Lipase frome R. miehei | rhizomucor miehei |
| Lipase frome T. launginosus | Thermomyces launginosus |
| Lipase frome T. launginosus mutant | Thermomyces launginosus |
| Lipase mutant, broad range | T. launginosus mutant |
| Lipase acceptin bulky substrate | fungal mutant |
| Lipase broad range | fungal |
| Lipase broad range | fungal mutant |
| Protease alkaline | bacillus clausii |
| Protease alkaline and temperature stable | bacillus hludurans |
| Protease alkaline | bacillus licheniformis |
| Protease | bacillus licheniformis |
| Protease | fusarium oxysporum |
| Protease | rhizomucor miehei |
| Lipase mucor sol | mucore miehei |
| Lipase mucor CF | mucore miehei |
| Lipase AS1 | Alcaligenese SPP |
| Lipase PF | pseudomonase fluresence |
| Lipase P1 | pseudomonase cepacia |
| Lipase CA(A) | candida antartica |
| Lipase PC | penicillium camebetrii |
| Lipase C2 | Candida cylindrecea |
| Lipase AN | Aspargilus niger |
| Lipase PS amono | pseudomonase stutzrei |
| Lipase AS2 | Alcaligenes SPP |
| Lipase A | Achromobeter SPP |
| Lipase RS | Rhizppus SPP |
| Lipase MM | mucore miehei |
| Lipase CA(B) | candida antartica |
| Lipase P2 | pseudomonase cepacia |
| Lipase C1 | Candida cylindrecea |
| Esterase BS3 | Bacillus species |

### Hydrolysis:

### Example 4

### Preparation of R—IBG-Me-ester (R-iso-butyl glutaric methyl ester)

To a 15 ml vial equipped with a magnetic stirrer were added at room temperature iso-butyl glutaric dimethyl-ester (“MG-di-Me-ester”) (240 mg, 1 mmol), buffer phosphate at pH 7 (15 vol, 3.6 ml) and an enzyme. The parameters and results of the reactions are summarized in the table below and the % of conversion was measured by HPLC relative to the amount of the starting in mole.

| Parameters | | | | Conver- | Optical |
|---|---|---|---|---|---|
| Enzyme | | T | t | sion | purity |
| Type | source | ° C. | h | (%) | R-Me-ester |
| lipase acceptin | fungal mutant | RT | 4 days | 24 | 90 |
| bulky substrate | | | | | |
| lipase frome R. miehei | rhizomucor miehei | RT | 4 days | 79 | 83 |
| lipase CA(A) | candida antartica | RT | 48 | 17 | 80 |
| lipase C2 | Candida cylindrecea | RT | 48 | 79 | 84 |
| lipase acceptin | fungal mutant | 38 | 48 | 65 | 90 |
| bulky substrate | | | | | |
| esterase BS3 | Bacillus species | RT | 48 | 15 | 79 |

### Example 5

### Preparation of R—IBG-Me-ester (R-iso-butyl glutaric methyl ester)

IBG-dimethyl ester (10.8 g) was suspended in 0.05 M potassium phosphate buffer (1000 ml) in a jacketed reactor equipped with pH probe and magnetically stirred. NZ51032 lipase from Aspergillus Oryzae (5 ml) was added. The mixture was stirred at 22-24° C. and for 72 h. The pH of the reaction was kept constant at 7.2 by addition of 1 M Na₂CO₃ solution (pH-stat). The mixture was extracted MTBE (100 ml), the resulting aqueous phase was acidified with concentrated HCl to pH 2.7 and then extracted with MTBE (3×150 mL). The combined organic phases were dried with MgSO₄ and evaporated to yield 9 g of colorless oil of R—IBG-Me-ester, 90% optical pure (89% yield).

### Example 6

### Preparation of S—IBG-Me-ester (S-iso-butyl glutaric methyl ester)

To a 15 ml vial equipped with magnetic stirrer were added at room temperature MG-di-Me-ester (240 mg, 1 mmol), potassium phosphate buffer at pH 7 (15 vol, 3.6 ml) and an enzyme. The parameters and results of the reactions are summarized in the table below and the % of conversion was measured by HPLC relative to the amount of the starting in mole.

| Parameters | | | | Conver- | Optical |
|---|---|---|---|---|---|
| Enzyme | | T | t | sion | purity |
| Type | source | ° C. | h | (%) | S-Me-ester |
| Lipase B | Candida antartica | RT | 4 days | 39 | 80 |
| Lipase B | Candida antartica | 38 | 48 | 23 | 82 |
| lipase B | Candida antartica | RT | 72 | 75 | 79 |
| lipase CA | Candida antartica | 38 | 48 | 54 | 80 |
| (isoformB) | | | | | |

### Example 7

### Preparation of S—IBG-Me-ester (S-iso-butyl glutaric methyl ester)

IBG-dimethyl ester (10.6 g) was suspended in 0.05 M potassium phosphate buffer (60 ml) and tert-pentanol (10 ml) in a jacketed reactor equipped with pH probe and magnetically stirred. The mixture was cooled to −2° C. and CaL-B liquid from Candida Antartica (1 ml, Novozymes; 7000 TBU/ml) was added. The reaction was stirred for 96 h, the pH of the reaction was kept constant at 7.3 by addition of 2 M NaOH (pH-stat). The mixture was extracted with MTBE (2×10 ml), the organic phase was extracted with NaHCO₃ (10 ml). The water-phase was acidified with conc. HCl to pH 1.5 and extracted with MTBE (3×10 ml). Evaporation of the combined extracts yielded 9.5 g colorless oil of S—IBG-Me-ester. 95.5% optical pure (96% yield).

### Example 8

### Preparation of S—IBG-Me-ester (S-iso-butyl glutaric methyl ester)

IBG-dimethyl ester (10.6 g) was suspended in 0.05 M phosphate buffer (60 ml) and tert-pentanol (10 ml) in a jacketed reactor equipped with pH probe and magnetically stirred. The mixture was cooled to −2° C. and Immozyme CaL-BY T2 from Candida Antartica (5 g, 6500 TBU/g) was added. The reaction was stirred for 76 h. The pH of the reaction was kept constant at 7.3 by addition of 2 M NaOH (pH-stat). The mixture was filtered to remove the Immozyme, followed by an extensive wash with water. The filtrate was extracted was acidified with conc. HCl to pH 1.5 and extracted with EtOAc (2×50 ml). Evaporation of the combined extracts yielded 9.1 g colorless oil of S—IBG-Me-ester, 95% optical pure (92% yield).

### Example 9

### Preparation of S—IBG-Me-ester (S-iso-butyl glutaric methyl ester)

IBG-dimethyl ester (10.6 g) was suspended in 0.05 M phosphate buffer (60 ml) and tert-pentanol (10 ml) in a jacketed reactor equipped with pH probe and magnetically stirred. The mixture was cooled to −2° C. and Immozyme Ca L-B T2 Candida Antartica (5 g, 2500 TBU/g) was added. The reaction was stirred for 96 h. The pH of the reaction was kept constant at 7.3 by addition of 2 M NaOH (pH-stat). The pH was added to 7.8 and incubation followed for another day. The mixture was filtered to remove the Immozyme, followed by 2 washings with water. The filtrate was acidified with conc. HCl to pH 1.5 and extracted with EtOAc (2×50 ml). Evaporation of the combined extracts yielded 9.46 g colorless oil of S—IBG-Me-ester, 95% optical pure (96% yield).

### Example 10

### Preparation of S—IBG-Me-ester (S-iso-butyl glutaric methyl ester)

IBG-dimethyl ester (10.6 g) was suspended in 0.05 M TRIS buffer (70 ml) and tert-pentanol (20 ml) in a jacketed reactor equipped with pH probe and magnetically stirred. The mixture was cooled to −2° C. and CaL-B liquid Candida Antartica (6 ml; 7000 TBU/ml) was added. The reaction was stirred for 40 h. The pH of the reaction was kept constant at 7.8 by addition of 2 M NaOH (pH-stat). The reaction mixture is ultrafiltered over a 5 kDa membrane using a Vivacell 250 ultrafiltration set-up (Sartorius, Aldrich Z629294) at 1.5 bar air pressure. About 100 ml filtrate was obtained, the retentate (about 25 ml) was stored overnight in the fridge and next day diluted once with water and filtered to a retentate volume of 33 ml. The combined 115 ml of filtrate was acidified with HCl to pH 1.5 and extracted with EtOAc (3×50 ml). The extracts were dried and evaporated to 8.7 g oil of S—IBG-Me-ester, 96% optical pure (88% yield).

### Example 11

### Preparation of S—IBG-Me-ester (S-iso-butyl glutaric methyl ester)

Phosphate buffer-50 mM (48 ml), tert-pentanol (8 ml) and Novozymes CaL-B liquid Candida Antartica (8 g) were charged into in a jacketed reactor equipped with pH probe and magnetically stirred. The mixture was cooled to −2° C. The pH of the reaction was kept constant at 7.2 by addition of 2.5 M NH₃ (pH-stat). The IBG-dimethyl ester (11.6 ml) was added drop wise at 0.25 ml/h, tert-pentanol (4 ml) was added at 0.1 ml/h. The reaction was stirred for 72 h, 99% conversion was obtained. The mixture was transferred to an ultrafiltration cell (Vivacell 250) and was ultrafiltered using 4 bar (air)-pressure. At 25 ml residue, the mixture was diluted once with water and ultrafiltered again. The combined filtrate was acidified and extracted with MTBE (75+50 ml). Dried on Na₂SO₄ and evaporated to give 10.2 g colorless oil of S—IBG-Me-ester, 96% optical pure (92% yield). The retentate of the ultrafiltration was transferred back to the jacketed vessel for re-use.

### Example 12

### Conversion of S—IBG Ester to R—CMH according to Example 21 of International Publication No. WO 2007/139933

A 50 ml three-neck-flask was charged with aqueous NH₃ 22% (25 ml, 8 vol.) and S—IBG-methyl ester (3.16 g). The solution was stirred at room temperature for 92 hours. 37% of HCl was added to obtain a pH of 3. The white slurry was cooled to 0° C., R—CMH was filtered and dried at 55° C. under vacuum during 14 hours to obtain 3.65 g of white powder R—CMH. (Optical purity—90%, Yield—100%).

### Example 13

### Conversion of R—IBG Ester to R—CMH According to Example 29 of International Publication No. WO 2007/139933

Step I: A round-bottomed flask is equipped with a magnetic stirrer and is charged with methylene dichloride (100 ml), (R)-3-(Methoxycarbonyl)methyl)-5-methylhexanoic acid (20 g) and with triethylamine (0.77 g) and cooled to 0-5° C. followed by addition of ethyl chloroformate (9 g). The mixture is stirred for 1-2 h at a temperature of 20° C. to 25° C., followed by quenching with 25% aqueous ammonia (100 ml). The resulted slurry is filtered and washed with water and dried to obtain a solid of (R)-methyl 3-(carbamoylmethyl)-5-methylhexanoate.

Step II: A flask is equipped with a magnetic stirrer and is charged with 3N HCl (100 ml) and (R)-methyl 3-(carbamoylmethyl)-5-methylhexanoate (20 g). The mixture is stirred for 1-10 hours at a temperature of 20° C. to 25° C., followed by quenching with 47% NaOH to pH 3. The resulting slurry is filtered, washed with water, and dried to obtain a white solid of (R)-3-(carbamoylmethyl)-5-methylhexanoic acid.

### Example 14

### Conversion of (R)—CMH to (S)-Pregabalin: Example 12 from U.S. Publication No. 2007/0073085

A reactor (0.5 L) was loaded with water (165 ml) and NaOH (35.5 g) to obtain a solution. The solution was cooled to 15° C. and (R)—CMH (33 g) was added. Br₂ (28.51 g) was added drop wise (15 min) while keeping the temperature below 25° C. The mixture was heated to 60° C. for 15 min and then cooled to 15° C. Iso-butanol was added (100 ml) and then a solution of H₂SO₄ (66%) (33 ml) was added. The phases were separated, and the aqueous phase was extracted with Iso-butanol (83 ml). To the combined organic phases Bu₃N (34.2 g) was added and the mixture was cooled to 2° C., and stirred for 2 hours. The solid was filtered, washed and dried at 55° C. under vacuum, providing (S)-Pregabalin.

### Example 15

### Conversion of R—IBG Ester to (R)-methyl 3-(carbamoylmethyl)-5-methylhexanoate

A three-necked flask equipped with an addition funnel, thermometer pocket, drying tube and a mechanical stirrer, is charged with acetone (125 ml), R—IBG ester (25 g, 0.086 mole), triethyl amine (10.43 g, 0.129 mole), and cooled to 0-5° C. followed by addition of pivaloyl chloride (12.43 g, 0.103 mole). The mixture is stirred for 1-2 hours at a temperature of 20° C. to 25° C., followed by quenching with 20% aqueous ammonia (250 ml). The resulted slurry is filtered and washed with water and dried to get (R)-methyl 3-(carbamoylmethyl)-5-methylhexanoate.

### Example 16

### Conversion of (R)-methyl 3-(carbamoylmethyl)-5-methylhexanoate to (S)-Pregabalin

A three-necked flask equipped with an addition funnel, thermometer pocket, drying tube and a mechanical stirrer, is charged with methanol (2000 ml), (R)-methyl 3-(carbamoylmethyl)-5-methylhexanoate,(200 g, 0.689 mole) and is cooled to 0° to 5° C. followed by addition of sodium methoxide (149 g, 2.758 mole). The reaction mass is cooled to −15 to −25° C. followed by addition of bromine (165.5 g, 1.034 mole) and stirred for 1-2 h at −15 to −25° C. The mixture is gradually warmed to a temperature of 0° C. and then to 55-65° C., followed by stirring for 1 to 2 hours. The solvent is then stripped off and water is added to the mass. The resulted slurry is further extracted with toluene, toluene layer washed with brine followed by stripping off the solvent. 4N hydrochloric acid (2580 ml), phenol (10.72 g, 0.114 mole), sodium chloride (78.15 g, 1.342 mole) is added to the mass and is heated to 105°-110° C. for 15-24 hours, and then cooled to room temperature, i.e., about 20° to about 25° C. An aqueous 40% sodium hydroxide solution is added in an amount sufficient to provide a pH of 1. The solution is then extracted with 600 ml of iso-butanol, the organic layer was separated, and Bu₃N is added in an amount sufficient to provide a pH of 4. The (S)-Pregabalin is precipitated, filtered, and washed with 100 ml of iso-butanol which on crystallization from isobutanol water mixture results in (S)-Pregabalin as white crystals.

### Example 17

### Preparation of 3-Isobutylglutaric Acid According to First Example of U.S. Pat. No. 5,616,793

A mixture of ethyl cyanoacetate (62.4 g), hexane (70 mL), isovaleraldehyde (52.11 g), and di-n-propylamine (0.55 g) was placed under reflux. Water was collected azeotropically using a water separator. When no additional water was being collected from the reaction, the reaction was cooled and subjected to vacuum distillation to remove the solvent. Diethyl malonate (105.7 g) and di-n-propylamine (5.6 g) were added to the remaining oil (primarily 2-cyano-5-methylhex-2-enoic acid ethyl ester). The mixture was stirred at 50° C. for 1 hour to form 2-cyano-4-ethoxycarbonyl-3-isobutylpentanedioic acid diethyl ester and then poured into an aqueous solution of hydrochloric acid (300 mL of 6N). The mixture was placed under reflux. The reaction was maintained under reflux until ¹H-NMR indicated that the hydrolysis and decarboxylation were complete (approximately 72 hours). The reaction was cooled to 70° C.-80° C. and the aqueous mixture was extracted with toluene (1×250 mL, 1×150 mL). The toluene extracts were combined and the solvent was removed by distillation to give 88.7 g of 3-isobutylglutaric acid as an oil. When purified 3-isobutylglutaric acid was a solid with a melting point in the range of about 40° C. to about 42° C. ¹H NMR (CDCl₃, 200 MHz): δ 0.92 (d, 6H, J=6.6 Hz), 1.23 (dd, 2H, J₁=6.6 Hz, J₂=6.5 Hz), 1.64 (m, 1H), 2.25-2.40 (m, 1), 2.40-2.55 (m, 4H). ¹³C NMR (CDCl₃): δ 22.4, 25.1, 29.5, 38.4, 43.4, 179.2 IR (KBr): 680.7, 906.4, 919.9, 1116.6, 1211.1, 1232.3, 1249.6, 1301.7, 1409.7, 1417.4, 1448.3, 1463.7, 1704.8, 2958.3, 3047.0 cm⁻¹.

We claim:

1. A process for preparing (S)-iso-butyl-glutaric ester or (R)-iso-butyl-glutaric ester having the formula 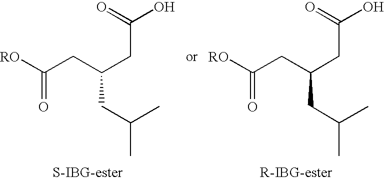
comprising: combining a suitable enzyme with a) 3-isobutylglutaric acid of the following formula 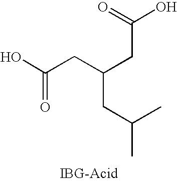
and an alcohol or an alkoxy donor that includes an OR group; or with b) 3-iso-butyl-glutaric diester of the following formula 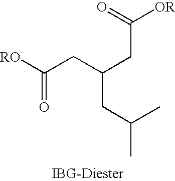
wherein the suitable enzyme is capable of stereoselectively esterifying 3-isobutylglutaric acid and stereoselectively hydrolyzing 3-iso-butyl-glutaric diester, respectively; and wherein R is a C₁₋₇ hydrocarbyl group.

2. The process of claim 1, wherein the process for preparing (S)-iso-butyl-glutaric ester or (R)-iso-butyl-glutaric ester having the formula 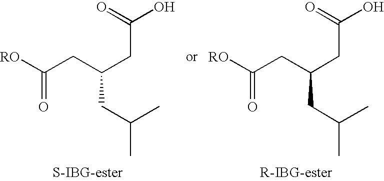
comprises: combining a suitable enzyme with 3-iso-butyl-glutaric diester of the following formula 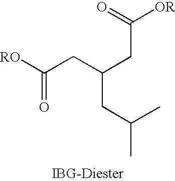
to obtain a reaction mixture; wherein the suitable enzyme is capable of stereoselectively hydrolyzing 3-iso-butyl-glutaric diester; and wherein R is a C₁₋₇ hydrocarbyl group.

3. The process of claim 1, wherein the process for preparing (S)-iso-butyl-glutaric ester or (R)-iso-butyl-glutaric ester having following formula 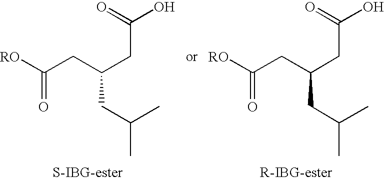
comprises: combining a suitable enzyme with 3-isobutylglutaric acid of the following formula 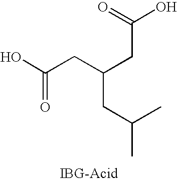
and an alcohol or an alkoxy donor that includes an OR group to obtain a reaction mixture, wherein the suitable enzyme is capable of stereoselectively esterifying 3-isobutylglutaric acid; and wherein R is a C₁₋₇ hydrocarbyl group.

4. The process of claim 2 or 3, wherein the C₁₋₇ hydrocarbyl group is methyl, ethyl, propyl, vinyl or n-butyl.

5. The process of claim 2 or 3, wherein the suitable enzyme is a hydrolase.

6. The process of claim 5, wherein the hydrolase is an esterase, protease or lipase.

7. The process of claim 6, wherein the esterase is selected from the group consisting of Esterase BS2 from bacillus species and Esterase BS3 from bacillus species.

8. The process of claim 6, wherein the lipase is selected from the group consisting of Lipase L-5, lipase from Aspergillus Oryzae, Lipase from Thermomyces lanuginosus, Lipase from Thermomyces lanuginosus mutant, Lipase mutant broad range from Thermomyces lanuginosus mutant, Lipase PS amono from Pseudomonas stutzeri, Lipase RS from Rhizopus spp., Lipase PF from Pseudomonas fluorescens, Lipase PC from Penicillium camenbertii, Lipase P1 from Pseudomonas cepacia, Lipase P2 from Pseudomonas cepacia, Lipase AN from Aspergillus niger, Lipase A from Candida Antartica, Lipase CA(A) from candida, Lipase CAL A from candida, Lipase AS1 from Alcaligenes spp., Lipase AS2 Alcaligenes spp, Lipase C2 from Candida cylindracea, Lipase C1 from Candida cylindracea, Lipase B from Candida Antartica, Lipase CA(B) from candida antartica, Lipase CAL B from candida Antartica, Lipase CAL B IM, Lipase from rhizomucor miehei, Lipase acceptin bulky substrate from fungal mutant, Lipase broad range from fungal, Lipase broad range from fungal mutant, Lipase mucor sol from mucore miehei, Lipase mucor CF from mucore miehei, and Lipase MM from mucore miehei.

9. The process of claim 6, wherein the protease is selected from the group consisting of Protease alkaline from bacillus clausii, Protease alkaline and temperature stable from bacillus hludurans, Protease alkaline from bacillus licheniformis, Protease from bacillus licheniformis, Protease from fusarium oxysporum, and Protease from rhizomucor miehei.

10. The process of claim 5, wherein the hydrolase is Lipase acceptin bulky substrate from fungal mutant, lipase from Aspergillus Oryzae, Lipase from rhizomucor miehei, Lipase B from Candida Antartica, Lipase CA(B) from candida antartica, Lipase CA(A) from candida antartica, Esterase BS3 from bacillus species, Lipase mucor sol from mucore miehei, Lipase C2 from Candida cylindracea, Lipase P2 from Pseudomonas cepacia, or Esterase BS2 from bacillus species.

11. The process of claim 2, wherein the reaction is done in the presence of a buffer that adjusts the reaction mixture to a pH suitable for the enzymatic activity.

12. The process of claim 2, wherein the reaction mixture further comprises a polar solvent.

13. The process of claim 12, wherein the polar solvent is a C₁₋₅ alcohol.

14. The process of claim 2, wherein the reaction further comprises recovering the obtained (S)-iso-butyl-glutaric ester or (R)-iso-butyl-glutaric ester from the reaction mixture.

15. The process of claim 3, wherein the alcohol or the alkoxy donor is a C₁₋₇ alcohol or C₁₋₇ alkoxy donor.

16. The process of claim 15, wherein the C₁₋₇ alcohol or the C₁₋₇ alkoxy donor is selected from the group consisting of a benzyl alcohol, methanol, ethanol, propanol, vinyl acetate, methyl acetate and n-butanol.

17. The process of claim 3, wherein the reaction mixture containing the enzyme, 3-isobutylglutaric acid and the alcohol or the alkoxy donor further contains a solvent.

18. The process of claim 17, wherein the solvent is selected from the group consisting of ketones, nitriles, aromatic hydrocarbons, ethers and mixtures thereof.

19. The process of claim 18, wherein the ketone is a C₃₋₆ ketone, the nitrile is a C₂₋₄ nitrile, the aromatic hydrocarbon is a C₆₋₉ aromatic hydrocarbon, and the ether is a C₃₋₇ ether.

20. The process of claim 19, wherein the C₃₋₆ ketone is acetone, methylethylketone, or methyl-isobutylketone, the C₂₋₄ nitrile is acetonitrile, the C₆₋₉ aromatic hydrocarbon is toluene, and the C₃₋₇ ether is diisopropylether, methyl-tertbutylether or tetrahydrofuran.

21. The process of claim 3, wherein the obtained (S)-iso-butyl-glutaric ester or (R)-iso-butyl-glutaric ester is recovered from the reaction mixture.

22. A process for preparing (S)-pregabalin comprising:
a) preparing (S)-iso-butyl-glutaric ester or (R)-iso-butyl-glutaric ester of the following formula; 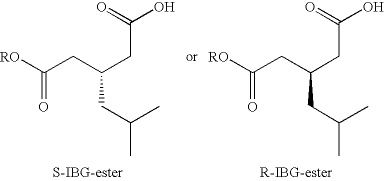
according to the process of claim 1; and
b) converting the (S)-iso-butyl-glutaric ester or (R)-iso-butyl-glutaric ester to (S)-pregabalin.

23. A composition comprising (S)-iso-butyl-glutaric ester and between 0.1% and 5% area by HPLC of (R)-iso-butyl-glutaric ester, based on the combined area % of said (R)-iso-butyl-glutaric ester and (S)-iso-butyl-glutaric ester as measured by HPLC.

24. The composition of claim 23 comprising (R)-iso-butyl-glutaric ester and between 95% and 99.9% area by HPLC of (S)-iso-butyl-glutaric ester, based on the combined area % of said (R)-iso-butyl-glutaric ester and (S)-iso-butyl-glutaric ester as measured by HPLC.

25. A composition comprising (R)-iso-butyl-glutaric ester and between 0.1% and 5% area by HPLC of (S)-iso-butyl-glutaric ester, based on the combined area % of said (R)-iso-butyl-glutaric ester and (S)-iso-butyl-glutaric ester as measured by HPLC.

26. The composition of claim 25 comprising (S)-iso-butyl-glutaric ester and between 95% and 99.9% area by HPLC of (R)-iso-butyl-glutaric ester, based on the combined area % of said (R)-iso-butyl-glutaric ester and (S)-iso-butyl-glutaric ester as measured by HPLC.
